(19) United States
(12) Patent Application Publication
(10) Pub. No.: US 2006/0110445 A1
(43) Pub. Date: May 25, 2006

(54) Dispersible tablet for oral administration

(76) Inventors: Shashikanth Isloor, Karnataka (IN); Shishir Bhand, Madhya Pradesh (IN); Sunilendu Bhushan, Maharashtra (IN); Rajiv Malik, Wein (AT)

Correspondence Address: RANBAXY INC., 600 COLLEGE ROAD EAST, SUITE 2100, PRINCETON, NJ, 08540, US

(21) Appl. No.: 10/521,423

(22) PCT Filed: Jul. 16, 2003
(86) PCT No.: PCT/IB03/02817

§ 371 Date: Jan. 18, 2006

(30) Foreign Application Priority Data
Jul. 16, 2002 (IN) 753/DEL/2002

(51) Int. Cl. A61K 9/20; A61K 31/43; A61K 31/545; A61K 31/407

(52) U.S. Cl. 424/464; 514/192; 514/200; 514/210.09

(57) ABSTRACT

The present invention relates to a process for the preparation of a dispersible tablet dosage form comprising β-lactam antibiotics for oral administration.

### FIELD OF THE INVENTION

The present invention relates to a process for the preparation of a dispersible tablet dosage form comprising β-lactam antibiotics for oral administration.

### BACKGROUND OF THE INVENTION

Beta-lactam antibiotics include penicillins like amoxicillin; cephalosporins like cefalexin, cefpodoxime proxetil, cefuroxime axetil, and cefaclor; carbapenems like loracarbef, imipenem, etc. have a broad spectrum of antibacterial activity against many gram-positive and gram-negative microorganisms. Effective average daily dosages of these antibiotics are typically quite high, and the film coated tablets produced to deliver the daily dose are large and often inconvenient to swallow by the very young or the elderly.

These dosage forms are also frequently not as bioavailable as aqueous suspension formulations which exhibit better bioavailability profiles. Bioavailability of the drug is one critical parameter for determining the efficacy of pharmaceutical formulations. The therapeutically effective amount of a medicine in a composition should be made available to the organism, with optimum blood concentrations of the active ingredients reached within the shortest possible time.

While the suspension dosage forms show the best bioavailability and can be easily administered to patients who have problems in swallowing, they have other drawbacks. They have to be reconstituted prior to administration and then stored under refrigerated conditions to prevent them from deterioration. Suspensions are also inconvenient to carry while traveling or when medication has to be taken away from home. They also involve the risk of inaccurate measurement and dosing.

There is therefore a need for dosage forms which have all the advantages of a tablet or capsule formulation and the bioavailability and convenience of administration of a suspension. A dispersible tablet is one such dosage form which meets the needs. They are easy to carry and can be reconstituted and administered to patients accurately and conveniently.

One of the key requirements of dispersible tablets is that they should disperse in an aqueous solution within a short time period of, for example, less than one minute, to form a smooth suspension without any coarse lumps.

U.S. Pat. No. 4,950,484 describes a dispersible tablet suited for amnphoteric beta-lactam antibiotic. U.S. Pat. No. 5,955,107 describes a pharmaceutical suspension tablet. U.S. Pat. No. 5,837,292 also describes fast, disintegrating and fast dissolving compositions marketed under the trade name Avicel® RC 501. U.S. Pat. Nos. 4,886,669 and 5,698,226 describe water dispersible tablet compositions containing swellable clays that generate high viscosity upon coming in contact with an aqueous solution. However, the use of swellable clays can undesirably retard the disintegration times of the tablet.

None of the prior art formulations provide a simple, easy to manufacture formulation for dispersible tablets. Further, to ensure patient compliance, the dispersible tablets should result in a suspension which has a smooth mouth feel without any gritty particles.

### SUMMARY OF THE INVENTION

Dispersible tablet formulations can be prepared using a simple formulation containing a single disintegrating agent without employing specific combinations of disintegrants, gum, etc.

In one aspect, there is provided a water dispersible tablet formulation including an active ingredient as beta lactam antibiotic, such as, for example, penicillin (e.g., amoxicillin), cephalosporin (e.g., cefuroxime axetil, cefpodoxime proxetil or cefalexin); or carbapenam (e.g., loracarbef or imipenem); and optionally a beta lactamase inhibitor, such as, for example, clavulanic acid or a salt thereof, such as potassium clavulanate; a disintegrating agent, such as, for example, croscarmellose sodium, polyvinylpyrolidone or sodium starch glycolate, said disintegrating agent being used both as intragranularly and extragranularly, and pharmaceutically accepted excipients.

If the disintegrant is used as an intragranular disintegrant, about 1% to about 2.5% w/w can be used. If the disintegrant is used as an extragranular disintegrant, about 1% to about 5% w/w can be used. The tablets can include a filler such as lactose, microcrystalline cellulose or starch, in about 40-70% w/w. The tablets can include lubricants such as talc, magnesium stearate, stearic acid or colloidal silicon dioxide.

The dispersible tablets can have a disintegration time of less than about one minute. The tablets can form a suspension after incorporating in water, for example, a suspension which passes through a 750 μm sieve.

If the formulation contains potassium clavulanate, the ratio of amoxicillin to potassium clavulanate can be, for example, from about 12:1 to about 1:1, or about 7:1.

The tablet, when dispersed in an aqueous media, can have a particle size distribution of, for example, d90 less than 600 μm, or d90 less than 400 μm, or d50 less than 300 μm.

Also provided herein, is a process for the preparation of a dispersible tablet including a beta lactam antibiotic (for example, 30-50% w/w amoxicillin, or amoxicillin with a particle size of d₉₀ less than about 150 μm, or less than about 75 μm), an optional beta lactamase inhibitor (for example, clavulanic acid or a salt thereof, such as potassium clavanulate) and an intragranular disintegrant, said beta lactam antibiotic, an optional beta lactamase inhibitor and said intragranular disintegrant (for example, about 1% to about 2.5% w/w of intragranular disintegrant) incorporated either in the dry mix or the granulating fluid, are aqueous granulated, dried (for example, dried to an equilibrium relative humidity of less than at 40% at a bed temperature of not more than 60° C., or for example, to an equilibrium relative humidity of less than 25% at a bed temperature of not more than 50° C.), mixed with extragranular disintegrant (for example, about 1% to about 5% w/w of extragranular disintegrant), a filler (for example, lactose, microcrystalline cellulose or starch, or, for example, filler in an amount of 40-70% w/w), a flavour, a lubricating agent (for example, talc, magnesium stearate, stearic acid or colloidal silicon dioxide), a sweetener and the resulting blend is compressed to tablets. Either disintegrant can be, for example, croscarmellose sodium, polyvinylpyrrolidone and sodium starch glycolate.

The dispersible tablets prepared this way can have a disintegration time of less than about one minute. The tablets can contain a ratio of amoxicillin to potassium clavulanate of about 12:1 to about 1:1, or about 7:1.

The process can be used to product tablets, that when dispersed in an aqueous medium, have particle size distribution of d90 less than 600 μm, or d90 less than 400 μm, or d50 less than 300 μm.

In another aspect, there is provided herein, a process for the preparation of a water-dispersible tablet formulation, the process including aqueous granulation of a β-lactam antibiotic and an intragranular disintegrant, incorporated either in the dry mix or in the granulating fluid; drying the granulated mixture; mixing the dried granules with optional extragranular disintegrants, fillers, flavours, sweeteners, or lubricating agents; and comprising the resulting blend to form water-dispersible tablets.

In another aspect, herein is provided a process for the preparation of a stable amoxicillin dispersible tablet formulation, the process including incorporating amoxicillin (for example, about 30 to about 50% w/w of the formulation, or for example, having a particle size of d₉₀ less than about 150 μm, or less than about 75 μm) and intragranular disintegrant (for example, croscarmellose sodium, polyvinylpyrrolidone, or sodium starch glycolate, for example, present in an amount of about 1% to about 2.5% w/w of the tablet formulation) are incorporated either in the dry mix or in the granulating fluid; drying the granulated mixture; mixing the dried granules with optional extragranular disintegrants (for example, croscarmellose sodium, for example, present in an amount of about 1 to about 5% w/w of the formulation), fillers (for example, lactose, microcrystalline cellulose, or starch, for example, present in an amount of about 40 to about 70% w/w), flavours, sweeteners, or lubricating agents (for example, talc, magnesium stearate, stearic acid, or colloidal silicon dioxide; and compressing the resulting blend to form water-dispersible tablets.

The process can be carried out wherein the granules are dried to an equilibrium relative humidity of less than about 40% at a bed temperature of not more than about 60° C., or less than about 25% at a bed temperature of not more than about 50° C. The dispersible tablet can have a disintegration time of less than about one minute. The suspension formed upon dispersion can desirably completely pass through a 750 μm sieve.

Amoxicillin granules may be further mixed with clavulanic acid or a salt thereof, for example, potassium clavulanate, in a ratio of amoxicillin to potassium clavulanate, for example, of about 12:1 to about 1:1, or about 7:1.

In another aspect, herein is provided a process for the preparation of a water-dispersible tablet formulation wherein the tablet when dispersed in an aqueous media, has a particle size distribution of d90 less than 600 μm, or less than about 400 μm, or the d50 is less than about 300 μm.

In another aspect, herein is provided a process for the preparation of a stable, dispersible tablet formulation of amoxicillin, and intragranular disintegrant, incorporated either in the dry mix or in the granulating fluid; drying the granulated mixture; mixing the dried granules with optional extragranular disintegrants, fillers, flavours, sweeteners, or lubricating agents; and comprising the resulting blend to form water-dispersible tablets, wherein the tablet is bioequivalent to the amoxicillin suspension formulation available commercially under the trade name Amoxil™ as required by the USFDA.

### DETAILED DESCRIPTION OF THE INVENTION

Water-dispersible tablet formulations are provided wherein the β-lactam antibiotic and an intragranular disintegrant are incorporated either in the dry mix or in the granulating fluid, are aqueous granulated, the granules are dried, mixed with extragranular disintegrant(s), fillers, flavours, sweeteners, lubricating agents and the resulting blend is then compressed to tablets.

Further, stable amoxicillin dispersible tablet formulations are provided, wherein the active ingredient and intragranular disintegrant are incorporated either in the dry mix or the granulating fluid, are aqueous granulated, dried, mixed with extragranular disintegrants, fillers, flavours, lubricating agents, sweeteners and the resulting blend is compressed to tablets.

Further, dispersible tablet formulations are provided wherein the tablet, when dispersed in an aqueous media, provides a suspension of five particles having a particle size distribution of d90 less than 600 μm.

Processes for the preparation of the above are also provided.

The β-lactam antibiotics used in accordance with the present invention can be, for example, penicillins, including amoxicillin; cephalosporins, including cefalexin, cefpodoxime proxetil, cefaclor and cefuroxime axetil; and carbapenems, including loracarbef, imipenem, and the like. Amoxicillin is a suitable β-lactam antibiotic.

The particle size of the β-lactam antibiotic suitable for the present formulations have d90 less than 150 μm. Also suitable are particles of size d90 less than 75 μm as measured by the Malvern laser diffraction method.

The β-lactam antibiotic can be present at a concentration of from about 30 to about 50% w/w of the formulation. The antibiotic can be granulated with an aqueous solution of a disintegrant. The disintegrant can be present intragranularly at a concentration of about 1% about 2.5% w/w of the tablet formulation.

The disintegrant used in accordance with the present invention can be superdisintegrants such as croscarmellose sodium, sodium starch glycolate, polyvinylpyrrolidone and the like. In some embodiments, the disintegrant can be croscarmellose sodium.

The process of wet granulation is suitable for the preparation of dispersible tablets, as it results in the formation of softer, more porous granules which can disintegrate in aqueous solution to give a smooth suspension, avoid the presence of coarse lumps. Amoxicillin and similar drugs are however, typically unstable when exposed to aqueous granulation. We have found that not only were the tablets of our formulation stable upon storage, they also had excellent disintegration characteristics, hardness and low friability.

The granules obtained from wet granulation are dried at a bed-temperature of less than about 60° C. to an equilibrium relative humidity of less than about 40%. Preferably, the granules are dried at a bed temperature of 50° C. to an equilibrium relative humidity of less than about 25%. The drying temperature is critical as amoxicillin degrades at higher temperatures. The dispersible tablets thus made showed excellent stability even under accelerated stability conditions of 40° C./75% relative humidity.

The size of the particles in the suspension is very important for a smooth mouth-feel. As per the British Pharmacopoeia, all the particles of a suspension should pass through a 710 μm sieve without leaving any residue. A suspension complying to this requirement can, however, still have a gritty mouth-feel. It is preferable, therefore to have a finer suspension containing a more uniform size particles. Dispersible tablets disclosed form a uniform dispersion upon swirling which has a smooth mouth feel and is free of gritty particles. The particle size distribution in the suspension is d90 less than 600 μm, for example, less than 400 μm. The d50 can be below 300 μm.

The granules thus prepared can be mixed with an extragranular disintegrant, a filler, a sweetening agent, pharmaceutically acceptable flavours, coloring agents and lubricants.

The amoxicillin granules may optionally be mixed with clavulanic acid or its salts. Preferably, the clavulanic acid salt used in the formulation is potassium clavulanate. The ratio of amoxicillin to potassium clavulanate used in accordance with this invention can be, for example, in the range from about 12:1 to about 1:1, for example, about 7:1.

The extragranular filler can be chosen from those commonly known in the art, for example, lactose and microcrystalline cellulose present at a concentration of between 40% to 70% w/w of the formulation. The extragranular disintegrant can be selected from the group comprising croscarmellose sodium, sodium starch glycolate, polyvinyl pyrrolidone and the like. In some embodiments, the intragranular and extragranular disintegrants are the same material. The disintegrant can be present at a concentration of between about 1 and about 5% w/w of the formulation.

The lubricants can be chosen from those commonly known in the art, for example, colloidal silicon dioxide, talc, stearic acid, magnesium stearate and the like.

The following examples further exemplify the invention and are not intended to limit the scope of the invention.

### Table 1

### EXAMPLES 1-6

TABLE 1
EXAMPLES 1-6

| | EXAMPLES | | | | | |
|---|---|---|---|---|---|---|
| DESCRIPTION | 1 | 2 | 3 | 4 | 5 | 6 |
| Intragranular | | | | | | |
| Loracarbef | — | — | — | — | — | 205 mg eq. |
| | | | | | | to 200 mg |
| | | | | | | loracarbef |
| Amoxicillin (as trihydrate) | 462.43 | 231.21 | 1010.80 | 693.12 | 231.0 | — |
| Croscarmellose sodium | 15.00 | 7.50 | 35.00 | 24.00 | 12.5 | 7.50 |
| Colour (Allura Red A1 Lake) | 0.50 | 0.25 | 0.50 | 0.34 | 0.50 | 0.25 |
| Purified Water | qs | qs | qs | qs | qs | qs |
| Extragranular | | | | | | |
| Potassium clavulanate + MCC(1:1) | — | — | — | — | 71.90 | — |
| eq to clav acid | | | | | 28.5 | |
| Croscarmellose sodium | 25.00 | 12.50 | 56.00 | 38.00 | 12.5 | 12.5 |
| Flavour | 10.00 | 10.00 | 20.00 | 20.00 | 20.0 | 10.0 |
| Colour (Allura Red A1 Lake) | 0.50 | 0.25 | 0.50 | 0.50 | 0.50 | 0.25 |
| Colloidal silicon dioxide | 10.00 | 5.0 | 21.00 | 15.00 | 5.0 | 5.0 |
| Aspartame | 10.00 | 10.00 | 20.00 | 20.00 | 10.0 | 10.0 |
| Microcrystalline cellulose | 451.57 | 215.79 | 1804.20 | 1227.88 | 200.0 | 215.79 |
| Magnesium stearate | 15.0 | 7.50 | 28.00 | 19.00 | 7.5 | 7.50 |
| Total Tablet Weight | 1000.00 | 500.00 | 2996.0 | 2058.00 | 600.00 | 500.00 |

Amoxicillin was granulated with an aqueous dispersion of croscarmellose sodium. The granules thus obtained were dried at a temperature of about 50-60° C. The equilibrium relative humidity (ERH) of the granules was NMT 40%. The dried granules were sized and blended with the remaining extragranular and compressed to tablets.

The column “205 mg eq. to 200 mg loracarbef” refers to the fact that 205 mg loracarbef monohydrate is equivalent to 200 mg of loracarbef anhydrous based on the following formula: [(200×100/100−water content)×100/assay on anhydrous data.] The water content of loracarbef monohydrate, per the U.S.P. is 3.5-6%. This gives the stated equivalence.

The dispersion prepared by suspending tablets made in accordance with Example 1 of this invention was subjected to a particle size analysis as measured by a Malvern laser diffractometer as given in Table 2.

TABLE 2
Particle size distribution of the suspension formed by
dispersing a tablet made in accordance with Example 1.

| | Particle size in μm |
|---|---|
| d90 | 110.0 |
| d50 | 37.0 |
| d10 | 8.7 |

The fine particles present in the suspension were uniformly distributed and resulted in an opaque suspension with negligible transmittance when scanned in a UV spectrophotometer at 200-800 nm.

A 400 mg dispersible tablet (made as per Example 1) was subjected to accelerated stability studies at 40° C. 175% RH as given in Table 3.

TABLE 3

| | | | | Related Substances | |
|---|---|---|---|---|---|
| | | | | (% w/w) | |
| | | | Dissolution | Individual | Total |
| | Assay | Fria- | (%) in 90 | Impurities | Impurities |
| Period | (mg) | bility | minutes | (NMT 1.0) | (NMT 4.0) |
| Initial | 401.1 | 0.1 | 103.1 | 0.226 | 0.782 |
| 1 Month | 399.0 | 0.2 | 101.9 | 0.168 | 0.963 |
| 2 Month | 397.4 | 0.2 | 99.7 | 0.212 | 0.907 |
| 3 Month | 397.2 | 0.2 | 100.7 | 0.150 | 1.002 |

As can be seen from the data given above the dispersible tablets made in accordance with the present invention displayed excellent stability characteristics under accelerated stability conditions of 40° C./75% even after 3 months.

A comparative, randomized two way crossover bioavailability study was conducted on an amoxicillin 400 mg dispersible tablet (as given in Example 1) formulation (test) and the commercially available Amoxil® (400 mg/5 ml) suspension formulation (reference) in twenty four healthy male volunteers under fasting conditions and the 90% confidence interval (T/R) and the ratio of least square means T/R (%) was calculated as given in Table 4.

TABLE 4

| | Cmax | AUC0-t | AUC 0-α |
|---|---|---|---|
| | (μg/ml) | (μg · h/ml) | (μg · h/ml) |
| 90% confidence | 85.3-94.1 | 93.7-98.8 | 93.9-99.0 |
| interval (T/R) | | | |
| T/R (%) | 89.6 | 96.2 | 96.4 |

As can be seen from the data, the dispersible tablets disclosed herein have a bioavailability profile very similar to that of the commercially available suspension formulation.

While embodiments herein have been described in terms of specific parameters, certain modifications and equivalents will be apparent to those skilled in the art and are intended to be included within the scope of the present invention.

1. A water dispersible tablet formulation comprising an active ingredient as beta lactam antibiotic and optionally a beta lactamase inhibitor, a disintegrating agent, said disintegrating agent being used both intragranularly and extragranularly, and pharmaceutically accepted excipients.

2. The formulation of claim 1 wherein said β-lactam antibiotic is selected from the group consisting of penicillin, cephalosporin and carbapenam.

3. The formulation of claim 1 wherein said penicillin is amoxicillin, said cephalosporins is cefuroxime axetil, cefpodoxime proxetil or cefalexin and said carbapenam is loracarbef or imipenem.

4. The formulation of claim 1 comprising the disintegrant selected from the group consisting of croscarmellose sodium, polyvinylpyrolidone and sodium starch glycolate.

5. The formulation of claim 1 comprising about 1% to about 2.5% w/w of an intragranular disintegrant.

6. The formulation of claim 1 comprising about 1% to about 5% w/w of an extragranular disintegrant.

7. The formulation of claim 1 comprising a filler selected from the group consisting of lactose, microcrystalline cellulose and starch.

8. The formulation of claim 1 further comprising 40-70% w/w of a filler.

9. The formulation of claim 1 comprising the lubricants selected from the group consisting of talc, magnesium stearate, stearic acid and colloidal silicon dioxide.

10. The formulation of claim 1 wherein said dispersible tablet has a disintegration time of less than one minute.

11. The formulation of claim 1 wherein said tablets form suspension after incorporating in aqueous media.

12. The formulation of claim 11 wherein said suspension formed completely passes through a 750 μm sieve.

13. The formulation of claim 1 wherein said beta lactamase inhibitor is clavulanic acid or a salt thereof.

14. The formulation of claim 13 wherein the clavulanic acid salt is potassium clavulanate.

15. The formulation of claim 14 wherein the ratio of amoxicillin to potassium clavulanate is 12:1 to 1:1.

16. The formulation of claim 15 wherein the ratio of amoxicillin to potassium clavulanate is 7:1.

17. The formulation of claim 11 wherein the tablet when dispersed in an aqueous media, has a particle size distribution of d90 less than 600 μm.

18. The formulation of claim 11 wherein the tablet when dispersed in an aqueous media, has a particle size distribution of d90 less than 400 μm.

19. The formulation of claim 11 wherein the tablet when dispersed in an aqueous media, has a particle size distribution of d50 less than 300 μm.

20. A process for the preparation of a dispersible tablet comprising a beta lactam antibiotic, an optional beta lactamase inhibitor and an intragranular disintegrant, the process comprising: aqueous granulating of a beta lactam antibiotic, an optional beta lactamase inhibitor and an intragranular disintegrant incorporated either in the dry mix or the granulating fluid; drying the granulation; missing the dried granulation with the extragranular disintegrant, a filler, a flavour, a lubricating agent, and a sweetener; and compressing the resulting blend into tablets.

21. The process of claim 20 wherein the tablet comprises 30-50% w/w amoxicillin.

22. The process of claim 21 wherein the amoxicillin has a particle size of d₉₀ less than 150 μm.

23. The process of claim 21 wherein the amoxicillin has a particle size of d₉₀ less than 75 μm.

24. The process of claim 20 wherein the tablet comprises about 1% to about 2.5% w/w of intragranular disintegrant.

25. The process of claim 20 wherein the tablet comprises about 1% to about 5% w/w of extragranular disintegrant.

26. The process of claim 24 wherein the disintegrant is selected from the group consisting of croscarmellose sodium, polyvinylpyrrolidone and sodium starch glycolate.

27. The process of claim 25 wherein the disintegrant is selected from the group consisting of croscarmellose sodium, polyvinylpyrrolidone and sodium starch glycolate.

28. (canceled)

29. (canceled)

30. (canceled)

31. The process of claim 20 wherein said granules are dried to an equilibrium relative humidity of less than at 40% at a bed temperature of not more than 60° C.

32. The process of claim 28 wherein said granules are dried to an equilibrium relative humidity of less than 25% at a bed temperature of not more than 50° C.

33. The process of claim 20 wherein said dispersible tablet has a disintegration time of less than one minute.

34. The process of claim 20 wherein the beta lactamase inhibitor is clavulanic acid or a salt thereof, and the beta lactam antibiotic is amoxicillin.

35. The process of claim 31 wherein the clavulanic acid salt is potassium clavulanate.

36. The process of claim 32 wherein the ratio of amoxicillin to potassium clavulanate is 12:1 to 1:1.

37. The process of claim 33 wherein the ratio of amoxicillin to potassium clavulanate is 7:1.

38. The process of claim 20 wherein the tablet when dispersed in an aqueous media, has a particle size distribution of d90 less than 600 μm.

39. The process of claim 20 wherein the tablet when dispersed in an aqueous media, has a particle size distribution of d90 less than 400 μm.

40. The process of claim 20 wherein the tablet when dispersed in an aqueous media, has a particle size distribution of d50 less than 300 μm.

41. A process for the preparation of a water-dispersible tablet formulation, the process comprising:
aqueous granulation of a β-lactam antibiotic and an intragranular disintegrant, incorporated either in the dry mix or in the granulating fluid;
drying the granulated mixture;
mixing the dried granules with optional extragranular disintegrants, fillers, flavours, sweeteners, or lubricating agents; and compressing the resulting blend to form water-dispersible tablets.

42. The process of claim 38, wherein the β-lactam antibiotic is selected from penicillins; cephalosporins; and carbapenems.

43. The process of claim 38, wherein the β-lactam antibiotic is amoxicillin.

44. The process of claim 38, wherein the disintegrant is selected from croscarmellose sodium, polyvinylpyrolidone, and sodium starch glycolate.

45. The process of claim 41, wherein the intragranular disintegrant is croscarmellose sodium.

46. The process of claim 41, wherein the disintegrant is present intragranularly at a concentration of about 1% to about 2.5% w/w of the tablet formulation.

47. (canceled)

48. (canceled)

49. (canceled)

50. (canceled)

51. (canceled)

52. (canceled)

53. The process of claim 38, wherein the suspension formed upon dispersion can completely pass through a 750 μm sieve.

54. A process for the preparation of a stable amoxicillin dispersible tablet formulation, the process comprising: granulation of amoxicillin and intragranular disintegrant; drying the granulated mixture; mixing the dried granules with optional extragranular disintegrants, fillers, flavours, sweeteners, or lubricating agents; and compressing the resulting blend to form water-dispersible tablets, wherein amoxicillin and intragranular disintegrant are incorporated either in the dry mix or in the granulating fluid.

55. The process of claim 45, wherein amoxicillin comprises about 30 to about 50% w/w of the formulation.

56. The process of claim 45, wherein amoxicillin has a particle size of d₉₀ less than about 150 μm.

57. The process of claim 45, wherein amoxicillin has a particle size of d₉₀ less than about 75 μm.

58. (canceled)

59. (canceled)

60. (canceled)

61. (canceled)

62. (canceled)

63. (canceled)

64. (canceled)

65. (canceled)

66. The process of claim 45, wherein the granules are dried to an equilibrium relative humidity of less than about 40% at a bed temperature of not more than about 60° C.

67. The process of claim 45, wherein the granules are preferably dried to an equilibrium relative humidity of less than about 25% at a bed temperature of not more than about 50° C.

68. (canceled)

69. (canceled)

70. (canceled)

71. (canceled)

72. (canceled)

73. (canceled)

74. The process of claim 45 wherein the tablet when dispersed in an aqueous media, has a particle size distribution of d90 less than 600 μm.

75. The process of claim 51, wherein the d90 is less than about 400 μm.

76. The process of claim 51, wherein the d50 is less than about 300 μm.

77. The process of claim 45 wherein the tablet is bioequivalent to the amoxicillin suspension formulation available commercially under the trade name Amoxil™ as required by the USFDA.
